(19) United States
(12) Patent Application Publication
(10) Pub. No.: US 2024/0362813 A1
(43) Pub. Date: Oct. 31, 2024

(54) METHOD FOR DETERMINING A VOLUME OF CALCIUM IN AN AORTA

(71) Applicant: Siemens Healthineers AG, Forchheim (DE)

(72) Inventors: Jonathan SPERL, Bamberg (DE); Saikiran RAPAKA, Pennington, NJ (US); Juraj SUTIAK, Bratislava (SK); Jana ORAVCOVA, Holic (SK)

(73) Assignee: Siemens Healthineers AG, Forchheim (DE)

(21) Appl. No.: 18/645,575

(22) Filed: Apr. 25, 2024

(30) Foreign Application Priority Data
Apr. 27, 2023 (DE) 102023203918.4

(51) Int. Cl. G06T 7/62; G06T 5/30; G06T 7/00; G06T 7/11; G06T 7/136; G16H 30/40

(52) U.S. Cl. CPC G06T 7/62; G06T 5/30; G06T 7/0012; G06T 7/11; G06T 7/136; G16H 30/40; G06T 2207/10081; G06T 2207/20032; G06T 2207/20081; G06T 2207/30101; G06T 2207/30172

(57) ABSTRACT

A computer-implemented method for determination of a volume of calcium in an aorta comprises receiving a medical image dataset of the aorta; determining an aorta center line of the aorta based on the medical image dataset; determining landmarks on the aorta based on the medical image dataset; determining an aorta mask of the aorta based on the medical image dataset; applying the aorta mask to the medical image dataset to create a masked medical image dataset; creating a calcium mask based on the masked medical image dataset; determining at least one aorta segment based on the aorta mask, the aorta center line and the landmarks; determining the volume of calcium of the at least one aorta segment based on the calcium mask and the at least one aorta segment; and providing the volume of calcium of the at least one aorta segment.

### CROSS-REFERENCE TO RELATED APPLICATION(S)

The present application claims priority under 35 U.S.C. § 119 to German Patent Application No. 10 2023 203 918.4, filed Apr. 27, 2023, the entire contents of which is incorporated herein by reference.

### FIELD

Independent of the grammatical term usage, individuals with male, female or other gender identities are included within the term.

One or more example embodiments of the present invention relates to a method for determining a volume of calcium in an aorta, to a determination apparatus for determination of a volume of calcium in an aorta, to a computer program product and to a computer-readable memory medium.

### RELATED ART

It is known that calcium or plaque deposit in arteries, for example in the aorta, and can lead to an increased risk of a cardio-vascular disease. This phenomenon is also referred to as calcification of an artery.

It is known that, for planning specific medical interventions on the cardio-vascular system, it is necessary to know about a calcification of the aorta. An intervention in a calcified part of an aorta can lead to an embolization. Calcified sections must also be taken into account when clamping-off the aorta. In this case it is moreover advantageous to know about the spatial distribution of the plaque in the aorta, since the clamping-off of the aorta at a calcified point is only possible with difficulty and hides further risks such as an embolization. For example, it is not possible or is only possible with difficulty to carry out a clamping-off on what is known as a porcelain aorta. A porcelain aorta is an aorta that has a marked, mostly circumferential, calcification. Typically, specifically for planning a medical intervention, a medical image dataset of the patient, which depicts an image of the aorta, is acquired. The medical image dataset typically comprises a computed tomography image. In this image the calcification of the aorta is analyzed manually, and the intervention is planned on the basis thereof. This is a time-consuming and thus also a cost-intensive process and means under some circumstances an additional computed tomography examination or acquisition of a further, new computed tomography recording for the patient.

It is moreover known that a calcification of the aorta or aorta plaque increases a risk of cardio-vascular disease. For this reason a systematic analysis of the calcification of the aorta for risk assessment and for early detection of cardio-vascular risks would be sensible and helpful. Typically such an analysis has to be carried out manually, which is why this type of risk assessment is mostly dispensed with, since it is too expensive and takes too much effort.

### SUMMARY

For at-risk patients in particular it would be sensible to determine the calcification of the aorta in order to detect an increased risk of a cardio-vascular illness at an early stage and initiate countermeasures where necessary. Typically there is already at least one medical image dataset of such at-risk patients, which has been acquired for a different purpose but still shows an image of the aorta. For example, for heavy smokers who have an increased cardio-vascular risk, lung screening for early detection of lung cancer is frequently carried out as standard. In this case a medical image dataset, which shows an image of the lungs, is recorded. Typically the aorta is also depicted in this medical image dataset. For cost reasons and on grounds of radiation protection it would be advantageous for the image data already available to be able to be employed for assessment of the cardio-vascular risk and for the calcification of the aorta to be able to be systematically detected from said data.

One or more example embodiments provides a method that makes possible an automated analysis of a calcification of an aorta in a medical image dataset of the aorta.

The object is achieved by the inventive method for determination of a volume of calcium in an aorta, by a determination apparatus for determination of a volume of calcium in an aorta, by a computer program product and by a computer-readable memory medium.

Inventive ways in which the object is achieved will be described below both in relation to the claimed apparatuses and also in relation to the claimed method. Features, advantages or alternate forms of embodiment are likewise also to be transferred to the other claimed subject matter and vice versa. In other words the physical claims (which are directed to an apparatus for example) can also be developed with features that are described or claimed in conjunction with a method. The corresponding functional features of the method are embodied in this case by the corresponding physical modules.

### BRIEF DESCRIPTION OF THE DRAWINGS

The characteristics, features and advantages of example embodiments described above will become clearer and easier to understand in conjunction with the following figures and their descriptions. In this case the figures and descriptions are not intended to restrict the invention and its forms of embodiments in any way.

In various figures the same components are provided with corresponding reference characters. As a rule the figures are not true-to-scale.

In the figures:

FIG. 1 shows a first exemplary embodiment of a method for determination of a volume of calcium in an aorta,

FIG. 2 shows a second exemplary embodiment of a method for determination of a volume of calcium in an aorta,

FIG. 3 shows a third exemplary embodiment of a method for determination of a volume of calcium in an aorta, and

FIG. 4 shows a determination apparatus for determination of a volume of calcium in an aorta according to one or more example embodiments.

### DETAILED DESCRIPTION

One or more example embodiments of the present invention relates to a computer-implemented method for determination of a volume of calcium in an aorta. The method comprises a step of receipt of a medical image dataset of an aorta. The method further comprises a step of determination of an aorta center line of the aorta based on the medical image dataset. The method further comprises a step of determination of landmarks on the aorta based on the medical image dataset. The method further comprises a step of determining an aorta mask based on the medical image dataset. The method further comprises a method step of application of the aorta mask to the medical image dataset. In this case a masked medical image dataset is created. The method further comprises a step of creating a calcium mask based on the masked medical image dataset. The method further comprises a step of determining at least one aorta segment based on the aorta mask, the center line of the aorta and the landmarks. The method further comprises a step of determination of the volume of calcium of the at least one aorta segment based on the calcium mask and the at least one aorta segment. The method further comprises a step of provision of the volume of calcium of the at least one aorta segment.

In the step of receipt of the medical image dataset the medical image dataset is received via an interface. The medical image dataset in this case can be provided directly from a medical imaging system or from a database, for example a Picture Archiving and Communication System (acronym: PACS).

The medical image dataset in this case comprises at least one recording or image of the aorta. The medical image dataset or the recording in this case comprises a plurality of image elements, especially of voxels or pixels, to which in each case an image value, especially a voxel value or pixel value, is assigned. The plurality of image elements is arranged in a matrix in this case.

In the step of determination of the center line of the aorta the center line of the aorta is determined based on the medical image dataset. The center line of the aorta is also described as the aorta center line. The center line of the aorta describes a central course of the aorta. The center line of the aorta is advantageously determined in an automated manner.

In the step of determination of landmarks of the aorta, landmarks on the aorta are determined. A landmark describes a point on the aorta or on the center line of the aorta. Landmarks can be determined for example with the aid of branching blood vessels and/or a specific shape of the aorta at a specific place. In particular standardized landmarks can be determined. As an alternative the landmarks can be determined as a function of a specific application case. For example for the case in which the aorta is to be clamped off, the landmarks are determined in such a way that they delimit the region of the aorta in which the clamping-off can be provided. The landmarks can be determined or specified manually. Advantageously the landmarks are determined in an automated manner.

In versions of one or more example embodiments of the present invention the landmarks can first of all be determined automatically and subsequently be supplemented and/or corrected via a user input. In other words at least one landmark can be supplemented by each user input for the automatically determined landmarks and/or at least one landmark determined automatically can be moved on the aorta via a user input.

In the step of determining the aorta mask the aorta mask is determined based on the medical image dataset. The mask describes which image elements of the medical image dataset image the aorta. The aorta mask can especially be a binary mask. The aorta mask in this case comprises a plurality of image elements that are arranged in the same way as the image elements of the medical image dataset. In other words the image elements of the aorta mask are arranged in a matrix with the same dimensions as the image elements of the medical image dataset. Thus an image element of the medical image dataset can be assigned to each image element of the aorta mask. Two image elements that are arranged at the same position or at positions that correspond to one another are assigned to each other in this case. Each image element of the aorta mask is likewise assigned an image value. The image values of the image elements of the aorta mask are either 0 or 1. The image elements of the aorta mask, which each correspond to an image element of the medical image dataset that images the aorta, have the image value 1. The other image elements of the aorta mask have the image value 0. As an alternative, instead of the image value 1, the image value “true” can be used and instead of the image value 0 the image value “false” can be used.

In the step of application of the aorta mask to the medical image dataset a masked medical image dataset is created. In particular, the aorta mask is applied to the recording included in the medical image dataset. In particular, on application of the aorta mask, the aorta mask is multiplied by the medical image dataset. In this case the image values of image elements corresponding to one another are multiplied by one another.

In the step of creating the calcium mask the calcium mask is created based on the masked medical image dataset. During creation of the calcium mask each image element of the masked medical image dataset within the masked region, i.e. within the aorta, is checked to see whether it shows calcium or not. If an image element shows calcium, its image value in the calcium mask is set to 1 or “true”. The image values of the other image element are set in the calcium mask to 0 or “false”. The calcium mask can be created in particular by application of a trained function to the masked image dataset. As an alternative the calcium mask can be created by application of a segmentation algorithm′ to the masked medical image dataset.

In the step of determination of at least one aorta segment, the aorta segment is determined based on the aorta mask, the center line of the aorta and the landmarks. An aorta segment comprises a part or a segment of the aorta. In other words the aorta segment comprises a section or a segment of the aorta in the masked medical image dataset. The center line of the aorta in this case can especially specify a direction along which the aorta segment extends. In this case the landmarks can specify the limits on the aorta between which the aorta segment extends. The aorta segment extends within the aorta mask. In other words the aorta mask predetermines a spatial limit of the aorta segment.

In the step of the determination of the volume of calcium of the at least one aorta segment, the volume of calcium in the at least one aorta segment is determined based on the calcium mask and the at least one aorta segment. For this the number of image elements with a value of 1 or “true” in the calcium mask within the at least one aorta segment can be determined. In this way the absolute volume of calcium in the at least one aorta segment is determined. Moreover, in versions of one or more example embodiments of the present invention the overall number of image elements in the aorta segment in the masked medical image dataset can be determined. Via the quotient of the number of image elements from the calcium mask and the overall number of the image elements in the aorta segment the proportion of calcium in the at least one aorta segment can be determined. The volume of calcium of the at least one aorta segment can thus be specified in the form of the absolute volume of calcium in the at least one aorta segment. As an alternative or in addition, the volume of calcium of the at least one aorta segment can be specified in the form of the (relative) proportion of calcium in the at least one aorta segment.

In the method step of provision of the volume of calcium of the at least one aorta segment, the volume of calcium of the at least one aorta segment is provided to a user and/or to another application. In particular the volume of calcium of the at least one aorta segment can be provided to a database, especially a PACS. As an alternative or in addition the volume of calcium of the at least one aorta segment can be displayed to a user via a display apparatus. The display apparatus can be a screen or a monitor, for example.

The inventors have recognized that, with the algorithm described, the volume of calcium of at least one aorta segment can be determined fully automatically. This makes possible a faster determination of the volume of calcium of at least one aorta segment. Thus it is possible to determine the volume of calcium of the at least one aorta segment “incidentally” without additional outlay. In this way an additional medically relevant factor for a risk assessment and/or intervention planning can be determined without additional outlay. It is not necessary to create or to acquire or to record a further medical image dataset simply for this purpose. It is sufficient for a medical image dataset that images the aorta to be available. This facilitates the determination of the volume of calcium of the at least one aorta segment and makes it possible to determine the volume of calcium of at least one aorta segment as standard without extra outlay when any given medical image dataset is acquired that images the aorta. The inventors have recognized that this knowledge improves a risk assessment for a coronary heart disease or for an embolism as described above. Moreover the inventors have recognized that intervention planning can be simplified and sped up in this way. Through there not being the necessity for an additional recording of a medical image dataset merely for the purposes of determining the volume of calcium, the radiation dose to which a patient is exposed is moreover reduced, when the medical image dataset is acquired with a radiation-based imaging. A radiation-based imaging is for example an x-ray imaging or a computed tomography imaging. Cost savings can also be made in this way. The inventors have moreover recognized that, through the determination of the volume of calcium of the at least one aorta segment, a spatial distribution of the volume of calcium can be determined. The spatial distribution is described by the distribution of the volume of calcium in various aorta segments. This spatial distribution is relevant in particular for intervention planning. The choice or the predetermination of the aorta segments enables the refinement or spatial resolution and the type of the spatial distribution to be predetermined. The inventors have moreover recognized that, through the automatic determination of the volume of calcium of the at least one aorta segment, a better comparability between various patients or between medical image datasets of a patient recorded at different times can be achieved than with a manual determination.

According to one or more example embodiments of the present invention the medical image dataset comprises a three-dimensional computed tomography recording.

The three-dimensional computed tomography (acronym: CT) recording depicts an image of the aorta. In particular the CT recording can be a recording of a lung screening. The image elements of the CT recording in this case are voxels and the image values are voxel values.

The inventors have recognized that the volume of calcium of the at least one aorta segment can be determined especially precisely in a three-dimensional CT recording. Moreover, the inventors have recognized that a corresponding CT recording is created as standard for a lung screening, which likewise records an image of the aorta. The inventors have recognized that, based on such a CT recording, the volume of calcium of the at least one aorta segment can be determined automatically without the acquisition of a further medical image dataset being necessary. This allows savings to be made in money and time as well as allowing the radiation dose to be reduced for the corresponding patient.

According to one or more example embodiments of the present invention the calcium mask is created via a threshold value segmentation of the masked image dataset.

In the step of creation of a calcium mask the calcium mask can be created or determined by a threshold value segmentation of the masked medical image dataset. The calcium mask is a binary mask. The calcium mask likewise comprises a plurality of image elements, which are arranged in a matrix with the same dimensions as the matrix of the medical image dataset or of the masked medical image dataset. Thus an image element of the calcium mask is assigned to each image element of the masked medical image dataset. In the threshold value segmentation, for all image elements of the calcium mask to which an image element of the masked medical image dataset with an image value above a predetermined threshold value is assigned, the image value of 1 is determined. For all image elements of the calcium mask to which an image element of the masked medical image dataset with an image value below the predetermined threshold value is assigned, the image value of 0 is determined. As an alternative, instead of the image value of 1, an image value of “true” and instead of the image value of 0 an image value of “false” can be determined. The threshold value is in particular predetermined. The threshold value in this case lies in particular between an image value of an image element that represents blood in the aorta and an image value of an image element that represents plaque or calcium. When the medical image dataset comprises a computed tomography recording, the image value for an image element that represents blood lies at around 0HU without contrast means and at around 600HU with contrast means. The image value of an image element that represents plaque then lies at around 1000HU. The threshold value is selected or predetermined in such a way that it lies between these values.

The inventors have recognized that a threshold value segmentation is a simple method for determination of the calcium mask. The inventors have recognized that, through the choice of the threshold value, the segmentation can be optimized in a simple manner.

According to a further aspect, the medical image dataset of the aorta comprises a recording of the aorta with contrast means.

In other words the medical image dataset can have been recorded without contrast means. The contrast means in this case is embodied to increase a contrast of the blood in the aorta in the medical image dataset.

The medical image dataset in this case can especially comprise a spectral computed tomography recording. A spectral computed tomography recording is also referred to as a multi-energy computed tomography recording or as a multi-energy recording. In a spectral computed tomography recording image characteristics can be changed via image processing. In particular, based on the spectral computed tomography recording, monoenergetic recordings can be created. Based on these monoenergetic recordings various materials in the recordings can be recognized and the recordings “decomposed” as a function of the materials. In this case a so-called material decomposition is carried out. In this way, in the monoenergetic recordings and/or in the spectral computed tomography recording, individual materials can be extracted, strengthened or removed. In particular in this way, based on a spectral computed tomography recording with contrast means, an artificial computed tomography recording without contrast means can be created. Based on this artificial computed tomography recording without contrast means the further steps of the method can be carried out. The recording of the aorta without contrast means can in this case be such an artificial computed tomography recording without contrast means.

The inventors have recognized that each recording of a medical image dataset for the determination of the volume of calcium of the at least one aorta segment can be used, regardless of whether it has or has not been recorded with contrast means. In this way it is no longer necessary to create a medical image dataset merely for the determination of the volume of calcium of the at least one aorta segment. This makes it possible also to determine the volume of calcium of the at least one aorta segment as part of a standard procedure and in this way to obtain a better risk assessment for coronary heart diseases without any additional costs or effort.

According to one or more example embodiments of the present invention the medical image dataset is a recording of the aorta without contrast means.

According to one or more example embodiments of the present invention, the method moreover comprises a step of dilation of the aorta mask.

In particular the dilation can be undertaken in the form of a morphological image operation on the aorta mask. Through the dilation the number of image elements in the aorta mask that have an image value of 1 or “true” will be increased. In other words the region in the aorta mask that specifies the location of the aorta in the matrix of the image elements will be enlarged. In particular the edges of the region with the image elements with image values of 1 or “true” will be smoothed by the dilation.

The dilation is in particular carried out or executed via a computing unit.

In an exemplary embodiment of the invention the dilated aorta mask replaces the aorta mask and instead of being carried out with the original aorta mask the previously described steps are carried out with the dilated aorta mask.

The inventors have recognized that the dilation makes sure that plaque or calcium which has deposited on the wall of the aorta is still arranged within the region of the aorta mask that specifies the location of the aorta or has the image values of 1 or “true”, and is taken into account in the creation of the calcium mask. Thus the proportion of calcium or the volume of calcium of the at least one aorta segment can be determined more precisely.

According to one or more example embodiments of the present invention the method moreover comprises a step of application of the dilated aorta mask to the medical image dataset, wherein a dilated masked medical image dataset is created. The method then further comprises a step of creation of a calcium mask based on the dilated masked medical image dataset. The method further comprises a step of determination of at least one dilated aorta segment based on the dilated aorta mask, the center line of the aorta and the landmarks. The method further comprises a step of determination of a dilated volume of calcium of the at least one dilated aorta segment based on the dilated calcium mask and the at least one dilated aorta segment. The method further comprises a step of selection of the volume of calcium of the at least one aorta segment to be provided between the volume of calcium of the at least one aorta segment and the dilated volume of calcium of the at least one dilated aorta segment. In the step of provision of the volume of calcium of the at least one aorta segment the selected volume of calcium of the at least one aorta segment is provided.

In other words the steps previously described are carried out in addition based on the dilated aorta mask. Two volumes of calcium are therefore determined for the at least one aorta segment, one based on the originally determined aorta mask and one based on the dilated aorta mask.

The volume of calcium that appears to better correspond to reality can then be selected in the step of selection. The selection can be made in this case by a user or automatically via an algorithm′. In this case a discrepancy between the two volumes of calcium determined of the at least one aorta segment can especially be taken into account. In particular account can be taken here of too great a discontinuity not occurring between the volume of calcium in two neighboring aorta segments.

In the step of provision of the volume of calcium of the at least one aorta segment, the selected volume of calcium is then provided.

The inventors have recognized that discrepancies can arise during determination of the volume of calcium through the dilation of the aorta mask. In this case, depending on the deposits of the calcium or plaque in the aorta, the dilated aorta mask can lead to better or worse results. The inventors have recognized that, without any extra effort, the volume of calcium can be determined based on both the non-dilated and also based on the dilated aorta mask. The inventors have recognized that in this case it can be determined or selected separately for each aorta segment which volume of calcium is to be provided. In this way it can be taken in account that the type or the location of the plaque deposits in the aorta can vary between the aorta segments.

According to a further aspect of one or more example embodiments of the present invention the method moreover comprises a step of filtering the calcium mask.

The filtering in particular enables holes in the calcium mask to be closed. As an alternative or in addition only scattered image elements recognized as showing plaque or calcium or only very small regions that have been recognized as plaque or calcium in the calcium mask can be filtered out. In other words the calcium mask is filtered in such a way that a specific number of image elements must depict a contiguous region in the calcium mask in order not to be filtered out. In particular the image values of scattered image elements or image element regions that have been set during determination of the calcium mask to 1 or “true” are set to 0 or “false”.

The filtering is carried out or executed especially via a computing unit.

The inventors have recognized that an influence of noise can be reduced by filtering the calcium mask. The noise can contribute to a falsification of the calcium mask and thus falsify the volume of calcium of the at least one aorta segment. The reduction of the influence of the noise enables the volume of calcium of the at least one aorta segment to be determined more precisely.

According to one or more example embodiments of the present invention the calcium mask is filtered with a median filter or a size filter.

The median filter or the size filter can be chosen so that, in regions of image elements that are smaller than a predetermined region, the image values are set to 0 or “false”.

The inventors have recognized that a median filter or a size filter are especially suitable for filtering the calcium mask in such a way that the noise can be reduced. In particular a median filter or a size filter can be adapted flexibly to the type of noise, in that the size of the regions to be filtered is adapted.

According to one or more example embodiments of the present invention the method furthermore comprises a step of classification of the volume of calcium of the at least one aorta segment wherein the class of the aorta segment is provided on provision of the volume of calcium of the at least one aorta segment.

In this case the volume of calcium of the at least one aorta segment comprises the proportion of calcium in the aorta segment. The classification is undertaken in this case especially based on the relative proportion of calcium of the at least one aorta segment. In this case, based on the proportion of calcium, the at least one aorta segment is sorted or classified into at least one of two classes. In other words, based on the proportion of calcium in an aorta segment, this aorta segment is assigned a class. A class in this case predetermines a range of the volume of calcium of an aorta segment or of the proportion of calcium in an aorta segment. An aorta segment of which the proportion of calcium lies within this range is assigned to this class. In particular at least two classes can be predefined. In particular three, four, five or six classes can be predefined. Predefined in this case means that the each of the classes is assigned to a corresponding range of the volume of calcium of an aorta segment or of the proportion of calcium. Advantageously the ranges of two classes do not overlap. Advantageously the ranges of two classes adjoin one another. For example a first class can predefine a range for a proportion of calcium of up to 28. A further class can then predefine a range for a proportion of calcium of more than 2%. Depending on the proportion of calcification ranges predetermined by the classes, the classes can then be assigned to a risk factor. For example one class can be “no risk”, a further class “moderate risk” and a third “high risk”.

The classification is in particular carried out or executed via a computing unit.

The inventors have recognized that the classification quickly and easily makes the information for the volume of calcium of the at least one aorta segment understandable. In this way the information can be reduced to the relevant statement. The inventors have recognized that in this way, without further extra effort, a rapid diagnosis about risk assessment for a coronary heart disease of a patient can be created. Moreover intervention planning can be simplified and sped up in this way.

According to one or more example embodiments of the present invention the volume of calcium of the at least one aorta segment is provided in the form of a DICOM Structured Report (acronym: DICOM SR) and/or in the form of a DICOM Secondary Capture (acronym: DICOM SC).

DICOM in this case is the acronym for Digital Imaging and Communications in Medicine.

The DICOM SR can be embodied in such a way that the volume of calcium of the at least one aorta segment can be integrated into a medical report.

The DICOM SC can be embodied in such a way that a user is able to be shown various aorta segments in various colors as a function of their volume of calcium or their proportion of calcium. In particular various aorta segments can be displayed in various colors based on the class assigned to them or colored in on a display. In other words the color of the various aorta segments in a display can depend on the respective class to which they are assigned.

For example, the at least one aorta segment or the aorta segments can be represented or displayed colored-in in traffic light colors. In this case an aorta segment with a volume of calcium that indicates a high risk can be shown in red and an aorta segment that only comprises a very small volume of calcium or no volume of calcium can be shown in green. An aorta segment with a volume of calcium that indicates a moderate risk can be shown in yellow.

The inventors have recognized that the volume of calcium of the at least one aorta segment can be integrated into a standardized report via the DICOM SR. Moreover the inventors have recognized that the volume of calcium of the at least one aorta segment can be represented in an easy-to-understand and clear manner via the DICOM SC. The inventors have recognized that in this way the volume of calcium of the at least one aorta segment can be provided in an accessible manner, so that the information can be acquired easily and quickly, and the additional information is available for a user without any extra outlay. Moreover, the information about the volume of calcium of the at least one aorta segment can be provided in a standardized form and thus be easily integrated into further applications.

According to one or more example embodiments of the present invention the at least one aorta segment extends along the center line of the aorta between two landmarks.

In other words the landmarks predetermine a limit for the at least one aorta segment. In particular, more than one aorta segment can be determined. Each of these aorta segments then extends between two landmarks. In this case two aorta segments adjoin one another in each case. The center line of the aorta in this case predetermines a direction of the at least one aorta segment.

The inventors have recognized that the aorta can be divided into aorta segments based on the landmarks. In particular a functional subdivision is possible in this case for example. Moreover a standardized and thus comparable subdivision of the aorta into aorta segments can be achieved via the landmarks. It can be predetermined in particular through the selection and the number of the landmarks how finely the spatial distribution of the plaque or calcium in the aorta is to be determined. The more landmarks are specified or determined, the finer is the spatial resolution of the plaque or calcium distribution in the aorta when a number of aorta segments are determined.

According to one or more example embodiments of the present invention the landmarks comprise at least one of the following landmarks: Aortic root, sinotubular transition, central ascending aorta, proximal aorta arch, central aorta arch, proximal descending aorta, central descending aorta, aorta at the diaphragm, abdominal aorta.

Alternative subdivisions, in particular also rough subdivisions by way of example in the ascending branch of the aorta, aorta arch and descending branch of the aorta are likewise possible.

The inventors have recognized that standardized landmarks can be used to subdivide the aorta into aorta segments. In this way it is more easily possible to compare the distribution of the plaque or calcium in the aorta with other parameters of the aorta, which are likewise determined for the at least one similar standardized aorta segment. Moreover, based on the standardized subdivision of the aorta into aorta segments, a better comparability between various patients and between various medical image datasets of a patient that were acquired at different points in time is possible.

According to one or more example embodiments of the present invention the method furthermore comprises the steps of creating intervention planning based on the volume of calcium of the at least one aorta segment and of provision of the intervention planning.

The intervention planning can be created manually or automatically. In particular preferred regions of the aorta for clamping off can be predetermined depending on the volume of calcium of the at least one aorta segment. As an alternative or in addition an aorta segment can be classified as at-risk if the volume of calcium, especially the proportion of calcium in the aorta segment is above a predetermined threshold value. The threshold value can for example be selected as a function of an embolization risk during an intervention.

The intervention planning creation is in particular carried out or executed via a computing unit.

In the step of provision of the intervention planning, the intervention planning is provided to a user or a database or to a medical engineering system via an interface.

In particular the intervention planning can be displayed to the user on a display apparatus. The display apparatus in this case can especially be a monitor or a screen. The user in this case can be a doctor or a medical assistant, for example.

As an alternative or in addition the intervention planning can be provided to a database, from which the intervention planning can be retrieved.

As an alternative or in addition the intervention planning can be provided to a medical engineering system. The medical engineering system can in this case for example be an operation robot or an operation support system.

The inventors have recognized that knowledge about the volume of calcium of the at least one aorta segment enables intervention planning to be supported. In particular in this way warnings for individual aorta segments can be output or preferred regions of the aorta for an intervention characterized or output. The inventors have recognized that in this way an intervention planning can be sped up and/or supported and the intervention itself can be carried out more safely as a result.

According to one or more example embodiments of the present invention the aorta mask is determined by applying a first trained function to the medical image dataset. As an alternative or in addition the center line of the aorta is determined by applying a second trained function to the medical image dataset. As an alternative or in addition the landmarks are determined by applying a third trained function to the medical image dataset.

In general a trained function imitates cognitive functions, that connect humans with human thinking. In particular through training based on training data the trained function can adapt itself to new circumstances and also recognize and extrapolate patterns.

In general parameters of a trained function can be adapted via training. In particular a supervised training, a semi-supervised training, an unsupervised training, a reinforcement learning and/or active learning can be used for this purpose. What is more, representation learning (an alternative term is feature learning) can be used. In particular the parameters of the trained functions can be adapted iteratively by a number of training steps.

In particular a trained function can comprise a neural network, a support vector machine, a decision tree and/or a Bayesian network, and/or the trained function can be based on k-means clustering, Q learning, genetic algorithms and/or association rules. In particular, a trained function can comprise a combination of a number of uncorrelated decision trees or an ensemble consisting of decision trees (random forest). In particular the trained function can be determined via XGBoosting (extreme Gradient Boosting). In particular a neural network can be a deep neural network, a convolutional neural network or a convolutional deep neural network.

Furthermore a neural network can be an adversarial network, a deep adversarial network and/or a generative adversarial network. In particular a neural network can be a recurrent neural network (recurrent neural network). In particular a recurrent neural network can be a network with long short-term memory (LSTM), especially a Gated Recurrent Unit (GRU). In particular a trained function can comprise a combination of the approaches described. In particular the approaches described here are mentioned for a trained function network architecture of the trained function.

The inventors have recognized that the method can be carried out fully automatically. The inventors have furthermore recognized that knowledge or experiences from earlier analyses of the aorta with which the trained functions have been trained can be used when carrying out the method. The inventors have recognized that algorithms already known can be applied for determining the aorta mask, the center line of the aorta and/or the landmarks.

One or more example embodiments of the present invention also relates to a determination apparatus for determination of a volume of calcium in an aorta. The determination apparatus comprises an interface and a computing unit. The interface is embodied for receipt of a medical image dataset of an aorta. The computing unit is embodied for determination of a center line of the aorta of the aorta based on the medical image dataset. The computing unit is furthermore embodied for determination of landmarks on the aorta based on the medical image dataset. The computing unit is furthermore embodied for determination of an aorta mask of the aorta based on the medical image dataset. The computing unit is furthermore embodied for application of the aorta mask to the medical image dataset. In this case a masked medical image dataset is created. The computing unit is furthermore embodied for creation of a calcium mask based on the masked medical image dataset. The computing unit is furthermore embodied for determination of at least one aorta segment based on the aorta mask, the center line of the aorta and the landmarks. The computing unit is furthermore embodied for determination of the volume of calcium of the at least one aorta segment based on the calcium mask and the at least one aorta segment. The interface is furthermore embodied for provision of the volume of calcium of the at least one aorta segment.

Such a determination apparatus can in particular be embodied to carry out the previously described method for determination of a volume of calcium in an aorta and its aspects. The determination apparatus is embodied to carry out this method and its aspects, in that the interface and the computing unit are embodied to carry out the corresponding method steps.

One or more example embodiments of the present invention also relates to a computer program product with a computer program as well as to a computer-readable medium. A largely software-based realization has the advantage that even a determination apparatus already used previously can be upgraded in a simple way by a software update in order to work in the way described. Such a computer program product, as well as the computer program, can if necessary have additional elements such as for example documentation and/or additional components, as well as hardware components, such as for example hardware keys (dongles etc.) for use of the software.

In particular one or more example embodiments of the present invention also relates to a computer program product with a computer program, which is able to be loaded directly into a memory of a determination apparatus, with program sections for carrying out all steps of the method described above for determination of the volume of calcium in an aorta and its aspects when the program sections are executed by the determination apparatus.

In particular one or more example embodiments of the present invention relates to a computer-readable memory medium, on which program sections that are able to be read and executed by a determination apparatus are stored for executing all steps of the method described above for determination of the volume of calcium in an aorta and its aspects when the program sections are executed by the determination apparatus.

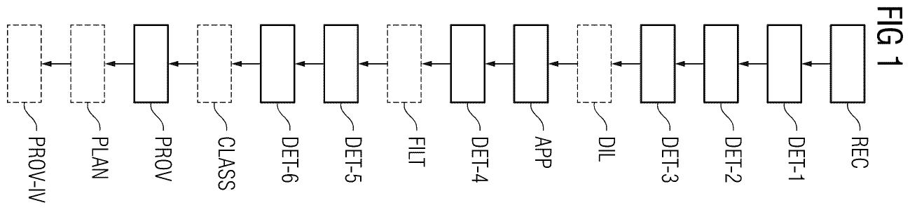

FIG. 1 shows a first exemplary embodiment of a method for determination of a volume of calcium in an aorta.

In a step of receipt REC of a medical image dataset IMG the medical image dataset IMG is received via an interface SYS.IF. The medical image dataset IMG can be provided by a medical imaging apparatus or a database for receipt REC. The database can for example be a Picture Archiving and Communication System (acronym: PACS). The imaging apparatus can for example be a computed tomography device, a magnetic resonance tomography device or an x-ray device.

The medical image dataset IMG comprises at least one recording that depicts the aorta of a patient. The medical image dataset IMG or the recording that it includes comprises a plurality of image elements, which are arranged in a matrix. The matrix can be two- or three-dimensional in this case. An image value is assigned to each image element in this case.

The medical image dataset IMG especially comprises a three-dimensional computed tomography recording. In particular the computed tomography recording can be a recording from a lung screening.

The medical image dataset IMG can have been acquired in particular with contrast means. A contrast means serves in this application to heighten a contrast of the blood in the aorta in relation to the environment, in the recording included in the medical image dataset IMG.

In this case the medical image dataset IMG advantageously comprises a spectral computed tomography recording. A spectral computed tomography recording is also referred to as a multi-energy computed tomography recording or as a multi-energy recording. Image characteristics can be changed in a spectral computed tomography recording via image processing. In particular monoenergetic recordings can be created based on the spectral computed tomography recording. Based on these monoenergetic recordings various materials can be recognized in the recordings and the recordings can be “decomposed” as a function of the materials. In this way, a so-called material decomposition is carried out. In this way individual materials can be extracted, strengthened or removed in the monoenergetic recordings and/or in the spectral computed tomography recording. In particular, based on the spectral computed tomography recording with contrast means, an artificial computed tomography recording can be created without contrast means. Based on this artificial computed tomography recording without contrast means the further steps of the method can be carried out.

As an alternative the medical image dataset IMG can have been acquired without contrast means.

In a further step of determination DET-1 of a center line of the aorta CL the center line of the aorta CL of the aorta is determined based on the medical image dataset IMG. The center line of the aorta CL is also referred to as the center line. The center line of the aorta CL describes the course of the aorta and runs along the center of the aorta.

The center line of the aorta CL can be determined in particular by application of a trained function to the medical image dataset IMG.

In a further step of determination DET-2 of landmarks LM, landmarks LM are determined on the aorta based on the medical image dataset IMG. The landmarks LM specify a location or a position on the aorta along the center line of the aorta AM.

In this case the landmarks LM can be general, standardized landmarks LM. For example, at least one landmark LM can be one of the following standard landmarks: Aortic root, sinotubular transition, central ascending aorta, proximal aorta arch, central aorta arch, proximal descending aorta, central descending aorta, aorta at the diaphragm, abdominal aorta.

As an alternative or in addition the landmarks LM can be application-specific. In other words the specific landmarks LM can depend on the purpose for which the volume of calcium of the at least one aorta segment is to be determined. For example, the region of the aorta that is relevant to the intervention can be restricted by the landmarks LM for intervention planning.

As an alternative or in addition the landmarks LM can be corrected or supplemented via a user input. A user can move or delete a landmark LM via a user input. As an alternative or in addition the user can insert a further landmark LM.

In versions of one or more example embodiments of the present invention the landmarks LM can be determined by applying a third trained function to the medical image dataset IMG.

In a further step of determination DET-3 of an aorta mask AM the aorta mask AM is determined based on the medical image dataset IMG. The aorta mask AM in this case is a binary mask. The aorta mask AM masks the aorta in the medical image dataset IMG or in the recording of the aorta included in this. The aorta mask comprises a plurality of image elements, which are arranged similarly to the image elements of the medical image dataset IMG. Thus an image element of the medical image dataset IMG is assigned to each image element of the aorta mask AM. The image elements of the aorta mask AM to which an image element in the medical image dataset IMG that depicts the aorta is assigned have an image value of 1 or “true”. All other image elements of the aorta mask have an image value of 0 or “false”.

In an optional step of dilation DIL of the aorta mask AM, the aorta mask AM is dilated via a morphological dilatation operator. In other words the aorta mask AM is enlarged at the edges of the region of the image elements with an image value of 1 or “true”. In other words the region in the aorta mask AM classified as aorta is enlarged. In the following steps of this exemplary embodiment the dilated aorta mask AM replaces the aorta mask AM determined originally.

In a further step of application APP of the aorta mask AM to the medical image dataset IMG, a masked medical image dataset IMG-mask is determined. On application APP of the aorta mask AM to the medical image dataset IMG, the medical image dataset IMG can be multiplied by the aorta mask AM. In particular, the recording included in the medical image dataset IMG can be multiplied by the aorta mask AM. In this case two image values of the aorta mask AM and of the medical image dataset IMG assigned to one another can be multiplied by one another in each case. The masked medical image dataset IMG-mask thus likewise comprises a plurality of image elements, which are arranged similarly to image elements of the medical image dataset IMG and of the aorta mask AM.

In a step of creation DET-4 of a calcium mask CM, the calcium mask CM is determined based on the masked medical image dataset IMG-mask.

In versions of one or more example embodiments of the present invention the calcium mask CM is created by a threshold value segmentation of the masked medical image dataset IMG-mask. In the threshold value segmentation a threshold value is specified or predetermined. All image values of the masked medical image dataset IMG-mask that lie below this threshold value are set in the calcium mask CM to 0 or “false”. All other image values of the calcium mask CM are set to 1 or “true”.

The threshold value in this case is embodied in such a way that it has a value between an average image value of the image elements that depict blood and an average image value of the image elements that depict plaque or calcium.

In a computed tomography recording without contrast means the value of an image value that depicts blood lies at approx. 0HU. The image values that depict calcium or plaque lie at appr. 1000HU. The threshold value typically lies between said values.

In a computed tomography recording with contrast means the value of an image value that depicts blood lies at up to approx. 600HU. The threshold value must also lie accordingly higher, in order to lie between 600 and 1000HU.

As an alternative the calcium mask CM can be created by application of a trained function to the masked medical image dataset.

In an optional step of filtration FILT the calcium mask CM is filtered. In particular the calcium mask CM is filtered via a size filter or a median filter. In this way only smaller contiguous regions in the calcium mask CM that have the image value 1 or “true” can be “erased” or filtered out. In other words a contiguous region in the calcium mask CM must comprise at least a predetermined number of image elements for it not to be filtered out and thus for it to be recognized as calcium deposits or plaque deposits. In this way noise that arises during the creation DET-4 of the calcium mask CM can be filtered or reduced.

In a further step of determination DET-5 of at least one aorta segment AS the at least one aorta segment AS is determined based on the aorta mask AM, the center line of the aorta CM and the landmarks LM. In particular the at least one aorta segment AS can extend between two landmarks LM. The center line of the aorta CL in this case specifies a direction along which the aorta segment AS extends. The aorta mask AM specifies the spatial extent of the aorta segment AS extending away from the center line of the aorta CL or limits said extent.

In versions of one or more example embodiments of the present invention more than one aorta segment AS can be determined. In particular the aorta can be divided into a number of aorta segments AS adjoining one another.

In a further step of determination DET-6 of the volume of calcium of the at least one aorta segment AS, the volume of calcium of the at least one aorta segment AS is determined based on the calcium mask CM and the at least one aorta segment AS.

For this the number of image elements with an image value of 1 or “true” that lie in the calcium mask CM within the aorta segment AS is determined. Optionally, the number of image elements within the aorta segment AS can in addition be determined based on the aorta mask AM or on the masked medical image dataset IMG-mask.

The volume of calcium of the at least one aorta segment AS can be specified as an absolute volume of calcium, in the form of the number of image elements within the aorta segment AS determined based on the calcium mask CM. The number of image elements can be converted into another unit, for example ml.

As an alternative or in addition the volume of calcium of the at least one aorta segment AS can be specified as a relative proportion of calcium. For this the quotient of the number of image elements from the calcium mask CM in the aorta segment AS and the number of image elements in the aorta segment AS in the aorta mask AM or the masked medical image dataset IMG-mask is determined. The quotient then specifies the relative proportion of calcium in the aorta segment AS.

The volume of calcium of the at least one aorta segment AS is determined for all aorta segments AS determined previously.

In an optional step of classification CLASS the volume of calcium of the at least one aorta segment AS is classified. In this case the volume of calcium is classified in particular when it was determined as a proportion of calcium. A class in this case predetermines a range of the proportion of calcium. The ranges of two classes are disjoint in this case. In particular the ranges of two or more classes can adjoin one another and jointly form a larger range. An aorta segment AS is assigned to a class when the volume of calcium or the proportion of calcium in this aorta segment AS lies within the range of the class. The classes can have various designations, which depict a risk factor for the corresponding volume of calcium within the class. For example a class can be designated “no risk” or “moderate risk” or “high risk”.

In a further step of provision PROV, the volume of calcium of the at least one aorta segment AS is provided. In particular the volume of calcium of the at least one aorta segment AS is provided via an interface SYS.IF. The volume of calcium of the at least one aorta segment AS can be provided to a user via a display unit, for example a monitor or screen. As an alternative or in addition the volume of calcium of the at least one aorta segment AS can be provided to a database, especially to a PACS.

In particular the class of the at least one aorta segment AS can be provided. In particular the classes of all aorta segments AS can be provided.

The volume of calcium of the at least one aorta segment AS can be provided in the form of a DICOM Structured Report (acronym: DICOM SR). In this way the volume of calcium of the at least one aorta segment AS can be integrated directly into a report.

As an alternative or in addition the volume of calcium of the at least one aorta segment AS can be provided in the form of a DICOM Secondary Capture (acronym: DICOM SC). In particular the volume of calcium can be provided in the form of the proportion of calcium of the at least one aorta segment AS. For example, a user can be provided with the volume of calcium of the at least one aorta segment AS by a color being assigned to each class and by a schematic aorta with the at least one aorta segment AS being displayed in the color of the assigned class. In this case for example “green” can be chosen for aorta segments with a volume of calcium or proportion of calcium below a first threshold value and thus with no risk. Aorta segments of which the volume of calcium or proportion of calcium lies above the first threshold value but below a second threshold value and which thus give rise to a moderate risk can be shown in yellow. Aorta segments of which the volume of calcium or proportion of calcium lies above the second threshold value, and which thus give rise to a high risk, can be shown in red. As an alternative or in addition the volume of calcium of the at least one aorta segment AS can be provided in the form of a table.

In an optional step of creation PLAN of an intervention planning, an intervention is planned based on the volume of calcium of the at least one aorta segment AS. In particular in this case aorta segments AS with a proportion of calcium above a predetermined threshold value can be identified as risk segments. As an alternative or in addition, the aorta segments AS or the aorta segment AS which have/has a proportion of calcium below the threshold value and is or are thus suitable for clamping-off, for example, can be determined as a function of the volume of calcium of the at least one aorta segment AS.

In the optional step of the provision PROV-IV, the intervention planning is provided. In this case the intervention planning can be provided to a user. For this the intervention planning can in particular be displayed to a user on a display apparatus, for example a screen or monitor. As an alternative or in addition the intervention planning can be provided to a database. The intervention planning can then be held in the database and retrieved as needed. As an alternative or in addition the intervention planning can be provided to a medical engineering system, for example an operation robot or an operation support system.

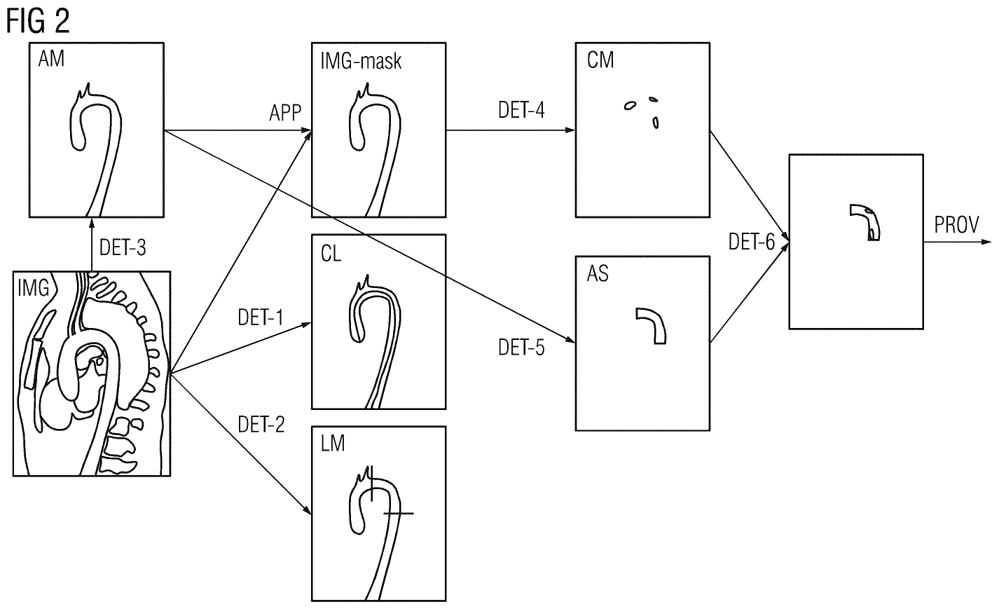

FIG. 2 shows a second exemplary embodiment of a method for determination of a volume of calcium in an aorta.

The second exemplary embodiment shows steps described in FIG. 1 on an example dataset.

Shown in FIG. 2 is a medical image dataset IMG. This medical image dataset IMG depicts the aorta. Based on the medical image dataset IMG, the aorta mask AM, the center line of the aorta CL and the landmarks LM will be determined. Two landmarks LM are specified here by way of example. The number and the position of the landmarks LM is however only regarded as an example. Based on the medical image dataset IMG and the aorta mask AM, the masked medical image dataset IMG-mask is determined. Then in its turn, as described in relation to FIG. 1, the calcium mask CM is determined based on the masked medical image dataset IMG-mask. The at least one aorta segment AS is determined based on the aorta mask AM, the center line of the aorta CL and the landmarks LM. Then, based on the calcium mask CM and the at least one aorta segment AS, as described in relation to FIG. 1, the volume of calcium in the aorta segment AS can be determined and provided.

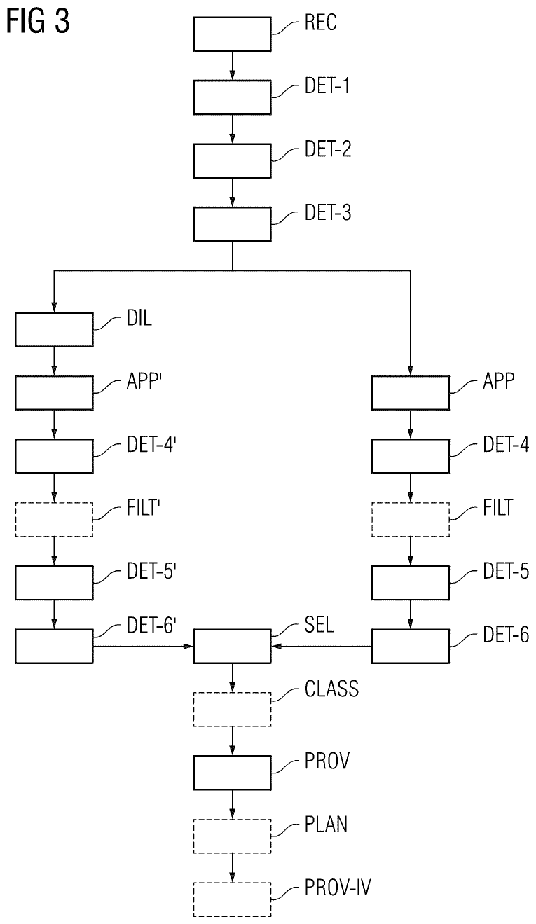

FIG. 3 shows a third exemplary embodiment of a method for determination of a volume of calcium in an aorta.

The steps described in relation to FIG. 1 are embodied in a similar way in the third exemplary embodiment.

The step of dilation DIL the aorta mask AM is not optional in this embodiment.

The dilated aorta mask does not replace the originally determined aorta mask AM in this exemplary embodiment. Instead the following steps are carried out both for the aorta mask AM originally determined and also for the dilated aorta mask. In this case the steps given below, and the similar steps described in relation to FIG. 1 for the originally determined aorta mask are carried out, wherein the dilated aorta mask is merely used instead of the originally determined aorta mask.

Thus, in a step of application APP′ of the dilated aorta mask, the dilated aorta mask is applied to the medical image dataset IMG. In this case a dilated masked medical image dataset is created.

In a step of creation DET-4′ of a dilated calcium mask the dilated calcium mask is created based on the dilated medical image dataset. In particular, as described in relation to FIG. 1, the dilated calcium mask is determined via a threshold value segmentation or by application a trained function to the dilated masked medical image dataset. In particular the dilated calcium mask can optionally be filtered as described above.

Based on the dilated aorta mask, the center line of the aorta CL and the landmarks LM, in a step of determination DET-5′ of at least one dilated aorta segment, at least one dilated aorta segment is determined. In this case the at least one dilated aorta segment advantageously extends between the same landmarks LM as the at least one non-dilated aorta segment AS. In particular a corresponding dilated aorta segment is determined for each aorta segment AS, wherein the two aorta segments corresponding to one another comprise the same sections of the aorta.

In a step of determination DET-6′ of a dilated volume of calcium of the at least one dilated aorta segment, the dilated volume of calcium of the at least one dilated aorta segment is determined based on the dilated calcium mask and the at least one dilated aorta segment.

In a further step of selection SEL of the volume of calcium of the at least one aorta segment to be provided, it is selected whether the volume of calcium of the at least one aorta segment AS or the dilated volume of calcium of the at least one dilated aorta segment is to be provided. The volume of calcium selected in this way is then provided for the at least one aorta segment or the corresponding at least one dilated aorta segment in the step of the provision PROV, as described in relation to FIG. 1. Furthermore the selected volume of calcium can be classified in the optional step of classification CLASS, as described in relation to FIG. 1.

During selection SEL it can especially be taken into consideration that the volume of calcium between two aorta segments adjoining one another is approximately constant.

If more than one aorta segment and thus also more than one dilated aorta segment has been determined, the selection SEL is carried out for each of the aorta segments and for the corresponding dilated aorta segments.

The further steps are again carried out as described in relation to FIG. 1 based on the selected volume of calcium of the at least one (dilated) aorta segment.

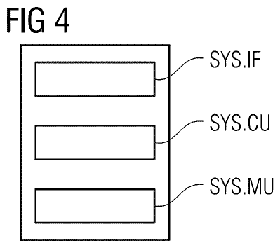

FIG. 4 shows a determination apparatus SYS for determination of a volume of calcium in an aorta.

The determination apparatus SYS shown for determination of a volume of calcium in an aorta is embodied to carry out an inventive method for determination of a volume of calcium in an aorta. The determination apparatus SYS comprises an interface SYS. IF, a computing unit SYS.CU and a memory unit SYS.MU.

The determination apparatus SYS can be in particular a computer, a microcontroller or an integrated circuit (IC). As an alternative the determination apparatus SYS can be a real or a virtual computer network (a technical term for a real computer network is “cluster”, a technical term for a virtual computer network is “Cloud”). The determination apparatus SYS can be embodied as a virtual system, which is executed on a computer or on a real computer network or on a virtual computer network (a technical term is “virtualization”).

The interface SYS. IF can be a hardware or software interface (for example a PCI bus, USB or Firewire). The computing unit SYS. CU can comprise hardware and/or software elements, for example a microprocessor or what is known as an FPGA (Field Programmable Gate Array). The memory unit SYS. MU can be embodied as a non-permanent working memory (Random Access Memory, RAM) or as permanent mass storage (hard disk, USB stick, SD card, Solid State Disk (SSD)).

The interface SYS. IF can comprise in particular a plurality of sub-interfaces, which carry out different method steps of the respective inventive method. In other words, the interface SYS. IF can be embodied as a plurality of interfaces SYS.IF. The computing unit SYS.CU can comprise in particular a plurality of sub-computing units, which carry out different method steps of the respective inventive method. In other words, the computing unit SYS. CU can be embodied as a plurality of computing units SYS.CU.

Where this has not yet explicitly occurred, but is however sensible and in the spirit of the invention, individual exemplary embodiments, individual of their sub aspects or features can be combined with one another or exchanged, without departing from the framework of the present invention. Advantages of the invention described in relation to one exemplary embodiment also apply, without this being explicitly stated, where transferrable, to other exemplary embodiments.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that encompasses a direct relationship relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A computer-implemented method for determination of a volume of calcium in an aorta, comprising:
receiving a medical image dataset of the aorta;
determining an aorta center line of the aorta based on the medical image dataset;
determining landmarks on the aorta based on the medical image dataset;
determining an aorta mask of the aorta based on the medical image dataset;
applying the aorta mask to the medical image dataset to create a masked medical image dataset;
creating a calcium mask based on the masked medical image dataset;
determining at least one aorta segment based on the aorta mask, the aorta center line and the landmarks;
determining the volume of calcium of the at least one aorta segment based on the calcium mask and the at least one aorta segment; and
providing the volume of calcium of the at least one aorta segment.

2. The method as claimed in claim 1, wherein the medical image dataset comprises a three-dimensional computed tomography recording.

3. The method of claim 1, wherein the calcium mask is created via a threshold value segmentation of the masked medical image dataset.

4. The method of claim 1, wherein the medical image dataset comprises a recording of the aorta with contrast means.

5. The method of claim 1, further comprising:
dilating the aorta mask.

6. The method of claim 5, further comprising:
applying the dilated aorta mask to the medical image dataset to create a dilated masked medical image dataset;
creating a dilated calcium mask based on the dilated masked medical image dataset;
determining at least one dilated aorta segment based on the dilated aorta mask, the aorta center line and the landmarks;
determining a dilated volume of calcium of the at least one dilated aorta segment based on the dilated calcium mask and the at least one dilated aorta segment; and
selecting the volume of calcium of the at least one aorta segment to be provided between the volume of calcium of the at least one aorta segment and the dilated volume of calcium of the at least one dilated aorta segment,
wherein the providing provides the selected volume of calcium of the at least one aorta segment.

7. The method of claim 1, further comprising:
filtering of the calcium mask.

8. The method of claim 5, wherein the calcium mask is filtered with a median filter or a size filter.

9. The method of claim 1, further comprising:
classifying the volume of calcium of the at least one aorta segment,
wherein the providing provides the class of the aorta segment.

10. The method of claim 1, wherein the providing provides the volume of calcium of the at least one aorta segment in a form of at least one of a DICOM Structured Report or a DICOM Secondary Capture.

11. The method of claim 1, wherein the at least one aorta segment extends along the aorta center line between two landmarks.

12. The method of claim 1, wherein the landmarks comprise at least one of the following landmarks:
an aortic root, a sinotubular transition, a central ascending aorta, a proximal aorta arch, a central aorta arch, a proximal descending aorta, a central descending aorta, an aorta at a diaphragm, or an abdominal aorta.

13. The method of claim 1, further comprising:
creating an intervention planning based on the volume of calcium of the at least one aorta segment; and
providing the intervention planning.

14. The method of claim 1, wherein at least one of
the determining the aorta mask includes a trained function to the medical image dataset,
the determining the aorta center line includes applying a second trained function to the medical image dataset, or
the determining the landmarks includes applying a third trained function to the medical image dataset.

15. A determination apparatus for determining a volume of calcium in an aorta, the determination apparatus comprising:
an interface configured to receive a medical image dataset of the aorta; and
a computing unit configured to cause the determination apparatus to,
determine an aorta center line of the aorta based on the medical image dataset,
determine landmarks on the aorta based on the medical image dataset,
determine an aorta mask of the aorta based on the medical image dataset,
apply the aorta mask to the medical image dataset to create a masked medical image dataset,
create a calcium mask based on the masked medical image dataset,
determine at least one aorta segment based on the aorta mask, the aorta center line and the landmarks, and
determine the volume of calcium of the at least one aorta segment based on the calcium mask and the at least one aorta segment,
wherein the interface is configured to provide the volume of calcium of the at least one aorta segment.

16. A computer program product with a computer program, when executed by a determination apparatus, cause the determination apparatus to perform the method of claim 1.

17. A computer-readable memory medium, on which program sections able to be read and executed by a determination apparatus are stored, and when executed by the determination apparatus, cause the determination apparatus to perform the method of claim 1.
